(19) United States
(12) Patent Application Publication
(10) Pub. No.: US 2006/0223768 A1
(43) Pub. Date: Oct. 5, 2006

(54) Regulation of gene expression

(76) Inventor: Jo Milner, York (GB)

Correspondence Address: ROBINS & PASTERNAK, 1731 EMBARCADERO ROAD, SUITE 230, PALO ALTO, CA, 94303, US

(21) Appl. No.: 10/549,711

(22) PCT Filed: Mar. 17, 2004
(86) PCT No.: PCT/GB04/01128

§ 371 Date: Jun. 5, 2006

(30) Foreign Application Priority Data
Mar. 18, 2003 (GB) 0306148.8

(51) Int. Cl. A61K 48/00; C12N 15/85; C07H 21/02

(52) U.S. Cl. 514/44; 435/455; 536/23.1

(57) ABSTRACT

The present invention relates to a method of regulating apoptosis. The method comprises the step of introducing into a cell an RNA construct comprising a nucleotide sequence which is homologous to mRNA within said cell. The mRNA within the cell includes genetic information of a gene element involved in the regulation of apoptosis. The invention also relates to an siRNA construct having a nucleotide sequence which is homologous to mRNA transcribed from a gene element involved in the regulation of apoptosis.

### FIELD OF THE INVENTION

This invention relates to the regulation of gene expression and in particular to the use of small interfering RNAs (siRNAs) in the regulation of apoptotic mediators.

### BACKGROUND TO THE INVENTION

The pathways governing apoptosis in mammalian cells are complex and the pro- and anti-apoptotic permutations regulating cell viability vary according to species, cell type, and also between normal and cancer cells (reviewed in Johnstone et al., 2002; Reed 2002; Cory and Adams, 2002). Imbalance in favour of cell survival enables tumour progression and resistance to anti-cancer drugs. For example, the pro-apoptotic Bax gene is frequently mutated in DNA mismatch repair-deficient tumours due to an unstable G8 tract at nucleotides 114 - 121 (Ionov et al., 1993; Rampino et al., 1997; Zhang et al., 200). When both Bax alleles are mutated the resultant Bax deficiency confers resistance to non-steroidal anti-inflammatory drugs (NSAIDs) such as sulindac and indomethicin (He et al., 1999; Yamamoto et al, 1999; Zhang et al., 2000).

Since predisposition to colorectal cancer is commonly associated with defective mismatch repair (Lynch, 1999), mutation in Bax may explain acquired resistance to sulindac when administered as chemopreventative agent. Indeed, sulindac enables clonal expansion of Bax-deficient cells in culture (Zhang et al., 2000) and may similarly favour clonal expansion of Bax-deficient cells in the colorectal epithelium of patients with inherited mismatch repair defects. However, Bax-deficient cells remain sensitive to 5-fluorouracil (5-FU) which activates p53-dependent apoptosis (Bunz et al., 1999; Zhang et al., 2000) and is the mainstream therapy for colorectal cancer.

There is a need to identify cellular pathways and apoptotic mediators that influence the survival of cancer cells. Such cellular pathways and apoptotic mediators represent promising targets for anti-cancer therapies.

The current model for mechanism of RNAi is based upon the observation that the introduced dsRNA is bound and cleaved by endonuclease RNase m to generate 21- and 22-nucleotide products. These small interfering RNAs (siRNAs) remain stably complexed with the endonuclease. The resulting dsRNA-protein complexes appear to represent the active effectors of selective degradation of homologous mRNA (Hamilton & Baulcombe, 1999; Zamore et al., 2000; Elbashir et al., 2001 a). Indeed, it has been established that duplexes of 21-nucleotide RNAs are sufficient to suppress expression in endogenous genes in mammalian cells (Elbashir et al., 2001b). This was demonstrated by selective silencing of endogenous lamin A/C expression in human epithelial cells following introduction of the cognate siRNA duplex. Thus, introduction of siRNA into mammalian cells is sufficient to selectively target homologous mRNA and silence gene expression. Importantly, siRNAs do not induce the non-specific interferon response, observed with dsRNAs>30 nucleotides long (Minks et al., 1979).

RNA interference is triggered by double-stranded RNA (dsRNA) and enables gene silencing by targeting specific mRNA transcripts for degradation: the silencing process is thus post-transcriptional and host cell genomic integrity is maintained. In mammalian cells RNA interference is induced by short interfering RNA (siRNA) duplexes (Elbashir et al., 2001). SiRNA duplexes target homologous mRNA for degradation with exquisite selectivity and very high, sustained efficacy. Moreover, gene silencing by a single dose of siRNA is achieved within a few days (Elbashir et al, 2001; Jiang and Milner, 2002) and avoids the need for protracted long-term selection procedures such as those necessary to establish gene knock-out cells. Thus RNA interference permits functional dissection of apoptotic pathways by silencing anti-apoptotic genes in cells in which specific pro-apoptotic genes are already deleted.

### STATEMENTS OF THE INVENTION

According to the present invention there is provided a method of regulating apoptosis, said method comprising introducing into a cell an RNA construct comprising a nucleotide sequence which is homologous to mRNA within said cell, wherein said mRNA includes genetic information of a gene element involved in the regulation of apoptosis.

Preferably the degree of homology of said RNA construct nucleotide sequence and said mRNA is at least 50% sequence identity, preferably at least 75%, more preferably at least 85% or at least 95% identity. Most preferably the degree of homology of said RNA construct nucleotide sequence and said mRNA is 92 to 95%, 97 to 99% or 100%. Preferably no more than 4 nucleotides of the RNA construct nucleotide sequence and the corresponding mRNA sequence are non identical. Preferably no more than 3, 2 or 1 nucleotides of the RNA construct nucleotide sequence and the corresponding mRNA sequence are non identical.

Preferably said gene element is involved in the suppression of apoptosis.

In a preferred embodiment of the invention said gene element is Bcl-2.

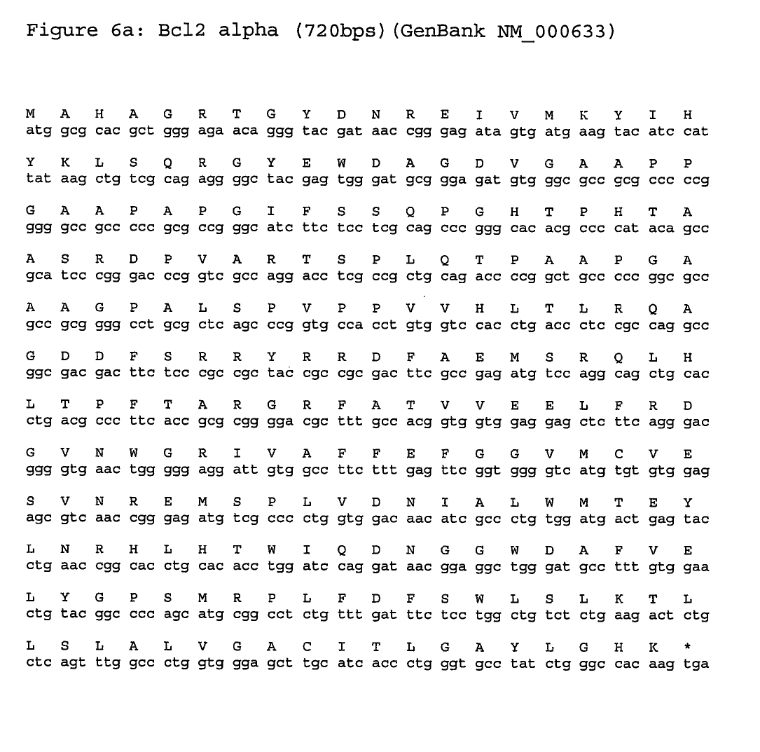

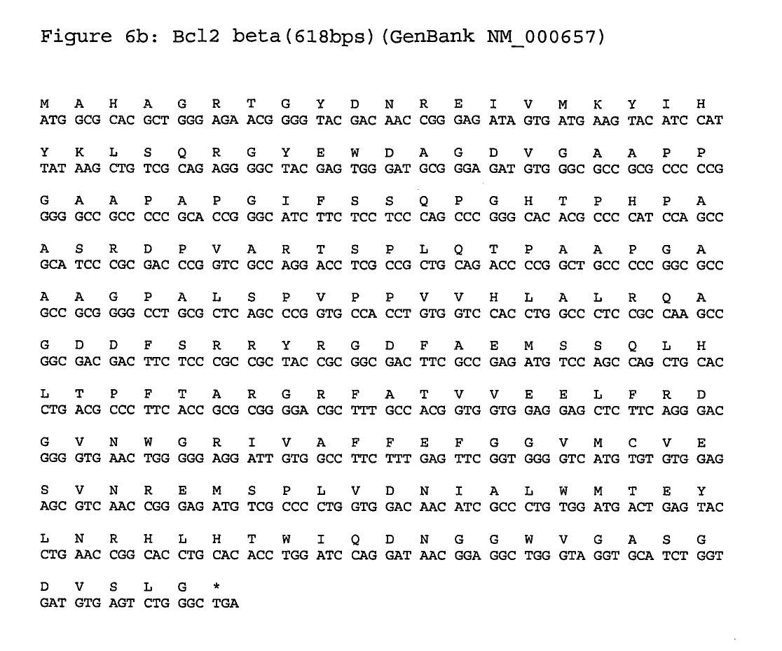

Preferably the gene element comprises a nucleic acid molecule, or part thereof, selected from the group consisting of:
(i) a nucleic acid molecule as represented by FIG. 6 or a functional fragment thereof;
(ii) a nucleic acid molecule which hybridises to any of the nucleic acid sequences in (i) and which has siRNA activity;
(iii) a nucleic acid molecule which is degenerate as a result of the genetic code to the nucleic acid sequence of (i) and/or (ii) above.

In a further preferred embodiment of the invention, said gene element is Bcl-x_{L}.

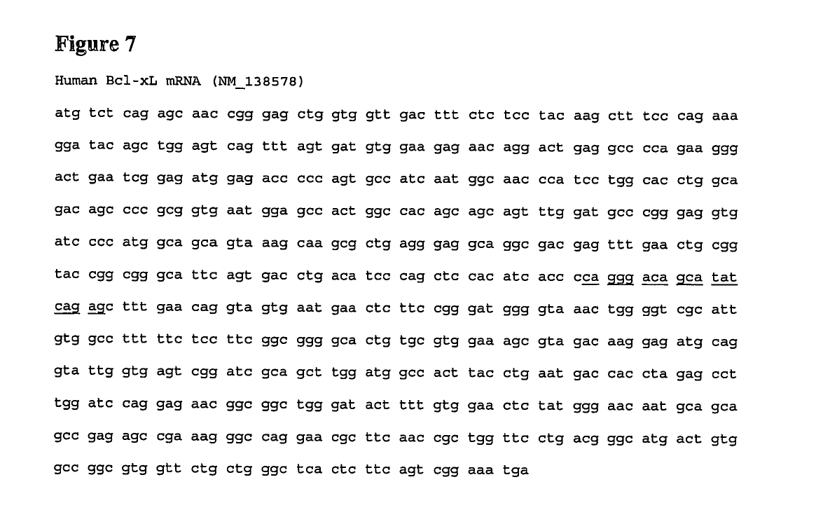

Preferably the gene element comprises a nucleic acid molecule, or part thereof, selected from the group consisting of:
(i) a nucleic acid molecule as represented by FIG. 7 or a functional fragment thereof;
(ii) a nucleic acid molecule which hybridises to any of the nucleic acid sequences in (i) and which has siRNA activity;
(iii) a nucleic acid molecule which is degenerate as a result of the genetic code to the nucleic acid sequence of (i) and/or (ii) above.

In a further preferred embodiment of the invention, said gene element is a viral homologue of a gene involved in the regulation of apoptosis.

The term viral homologue is used herein to refer to either a structural or functional homologue wherein the viral gene may have a certain degree of structural homology to an anti-apoptopic gene such as Bcl-2 or Bcl-x_{L}. Preferably the degree of homology is at least 50% sequence identity, more preferably at least 75% sequence identity, more preferably at least 85% identity and more preferably at least 95% sequence identity. Alternatively or additionally, said homologue may be a functional homologue of an anti-apoptopic gene wherein said homologue has a role in the regulation, preferably suppression, of cellular apoptosis.

Silencing of Bcl-2 was found to induce massive p53-dependent apoptosis and to occur under normal cell growth conditions (i.e. without recourse to genotoxic drugs necessary to activate p53 as a transcription factor).

The present invention concerns a novel pro-apoptotic function of p53 under Bcl-2 regulation, thus creating a constitutive Bcl-2/p53 axis regulating apoptosis in human colorectal epithelial cells.

Further experiments using isogenic clones of Bax± and Bax−/− cells and caspase 2 siRNA, clearly demonstrate that both Bax and caspase 2 are essential mediators of the Bcl-2/p53 apoptotic pathway. (Bax ± is a HCT116 derived cell line which has Bax gene, Bax−/− is a HCT116 derived cell line in which Bax gene has been knocked out).

The present invention also provides an siRNA construct having a nucleotide sequence which is homologous to mRNA transcribed from a gene element involved in the regulation of apoptosis.

Preferably the siRNA construct is from 15 to 25 nucleotides in length. More preferably the siRNA construct is from 19 to 23 nucleotides in length.

Preferably the siRNA construct comprises;
(i) a nucleotide sequence that is homologous to a part or fragment of the nucleic acid sequences in FIG. 6;
(ii) a nucleotide sequence which is degenerate as a result of the genetic code to the nucleic acid sequence of (i) above.

In a preferred embodiment of the invention said siRNA construct hybridises to the nucleic acid sequence in FIG. 6 under stringent hybridisation conditions. (Sambrook et al., (1989) Molecular Cloning; A Laboratory Approach).

Preferably the siRNA construct comprises a nucleotide sequence that is homologous to Bcl-2 mRNA nucleotides 354-372.

In an alternative embodiment of the invention the siRNA construct comprises;
(i) a nucleotide sequence that is homologous to a part or fragment of the nucleic acid sequence in FIG. 7;
(ii) a nucleotide sequence which is degenerate as a result of the genetic code to the nucleic acid sequence of (i) above.

In a preferred embodiment of the invention said siRNA construct hybridises to the nucleic acid sequence in FIG. 7 under stringent hybridisation conditions.

Preferably the siRNA construct comprises a nucleotide sequence that is homologous to BC1-x_{L} nucleotides 347-366.

In a further embodiment the invention also provides a method of treating a disease or condition associated with inappropriate apoptosis comprising administering to a subject an RNA construct wherein said RNA construct has a nucleotide sequence which is homologous to mRNA present within a cell of said subject and wherein said mRNA includes genetic information of a gene element involved in the regulation of apoptosis.

The term ‘inappropriate apoptosis’ is used herein to refer to the situation wherein the level of apoptosis is imbalanced in an undesirable way, for example, towards cell survival, which enables tumour progression and resistance to anti-cancer drugs.

In another embodiment the invention provides for the use of an RNA construct in the regulation of apoptosis in a cell, wherein said RNA construct has a nucleotide sequence which is homologous to mRNA within the cell and wherein said mRNA includes genetic information of a gene element involved in the regulation of apoptosis.

Accordingly the present invention provides an RNA construct for use as a medicament.

The present invention also provides for the use of an RNA construct for the manufacture of a medicament for the treatment of a disease or condition associated with inappropriate apoptosis.

The present invention also provides for the use of an RNA construct for the manufacture of a medicament to induce apoptosis.

In one embodiment of the invention the disease or condition associated with inappropriate apoptosis is colorectal cancer.

In an alternative embodiment of the invention, the disease or condition associatied with inappropriate apoptosis is a viral induced cancer.

In an alternative embodiment of the invention, the disease or condition associated with inappropriate apoptosis is colon cancer, leukaemia, breast cancer, cervical cancer, lung cancer, prostate cancer, lymphomas, liver cancer, renal cell carcinoma, neuroblastoma, adenocarcinoma, pancreatic cancer, malignant melanoma, uterine leiomyomas.

### DETAILED DESCRIPTION OF THE INVENTION

The present invention will now be described by way of example only and with reference to the following diagrams;

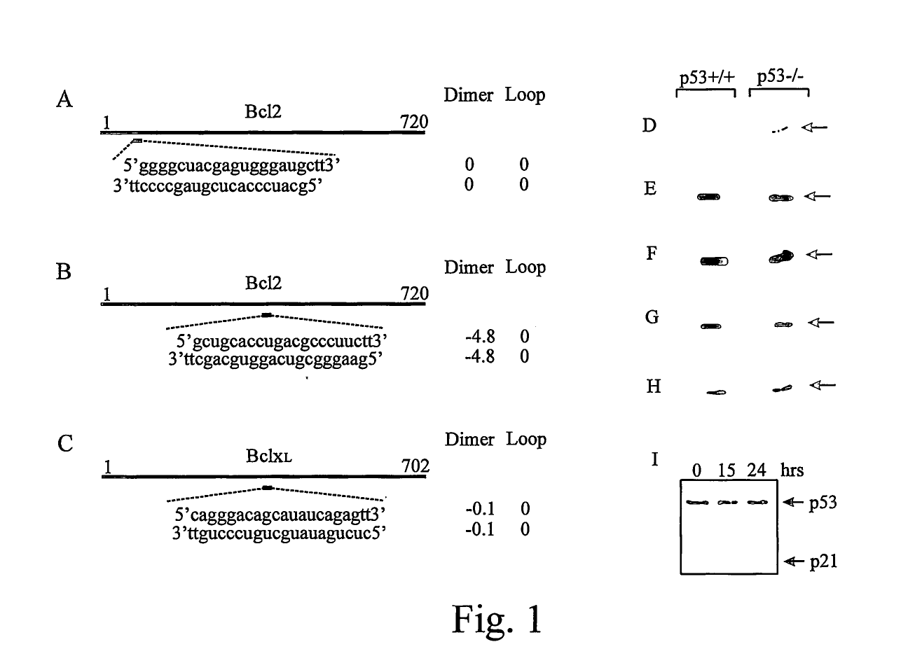

FIG. 1 illustrates the siRNA sequences used, and expression of Bcl-2 in HCT116 cells. a and b, Bcl-2 siRNA sequences, equivalent to Bcl-2 mRNA nucleotides 77-95 and 354-372 respectively; and c, BCl-x_{L} siRNA sequence, nucleotides 347-366 (See WO 03/008573 for methods of RNA production). Predicted secondary structures with propensity for base-pairing out of register (dimers) or for forming stem-loop structures (loops) were derived using Vector NTI. Anti-sense RNA controls employed Bcl-2 anti-sense nucleotides 354-372, and Bcl-x_{L} anti-sense nucleotides 347-366. Control siRNA (Jiang and Milner, 2002) and lamin A/C siRNA (Elbashir et al., 2001) were also used in this study. d-h, Immunoblots of Bcl-2 protein (arrowed) in HCT116 p53+/+ and p53−/− cell lysates using different anti-Bcl-2 antibodies: d, N19, Santa Cruz; e, =C-2, Santa Cruz; f, =Ab-1, Oncogene; this antibody gave non-specific cross-reactivity with multiple cellular proteins (not shown); g, =Ab-2, Oncogene; and h, =BD Pharmingen. The C-2 antibody (Santa Cruz) was used in all subsequent experiments. i, Immunoblots of p53 and p21 in HCT116 p53+/+ cells at different times after transfection with control siRNA as indicated.

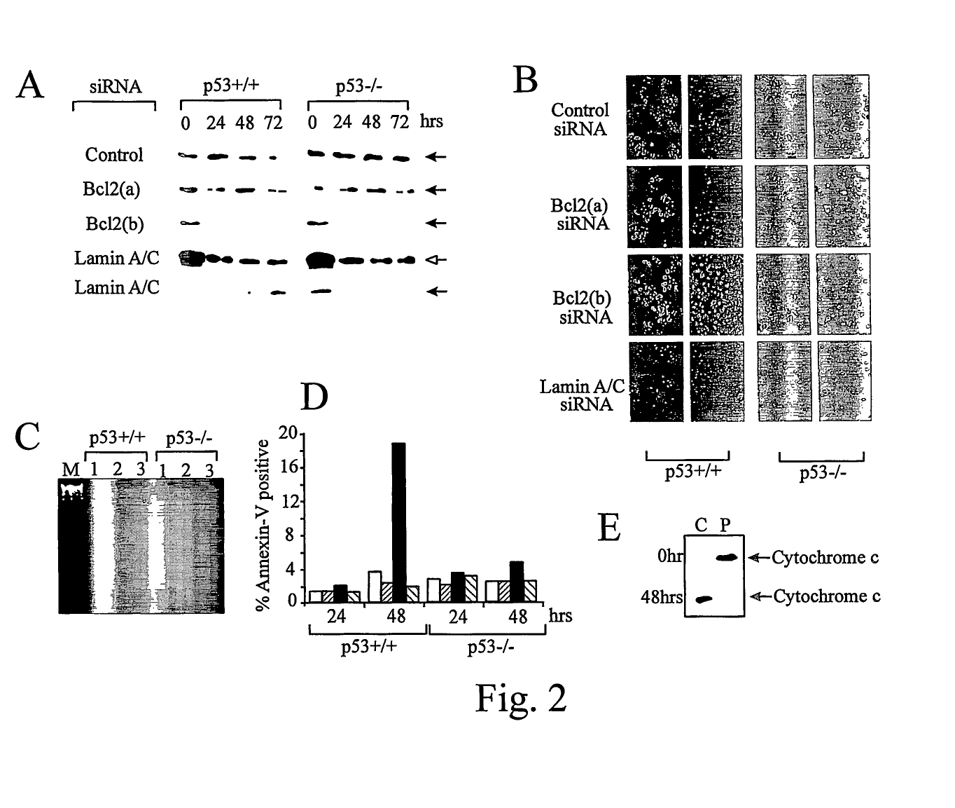

FIG. 2 illustrates siRNA silencing of Bcl-2 induces p53-dependent apoptosis. Isogenic clones of p53+/+ and p53−/− HCT116 cells were cultured and transfected with siRNAs as described previously (Methods). Transfection efficiency=70 -80%. a, Immunoblots of Bcl-2 (closed arrows), and lamin A/C (open arrow). Times post-transfection with control siRNA, Bcl-2 siRNAs and lamin A/C siRNA are as indicated. b, Phase contrast images of p53+/+ and p53−/− HCT1 16 cells at 24 and 48 hr post-transfection with control siRNA, Bcl-2 siRNAs or with lamin A/C siRNA. c, Apoptotic cells confirmed by DNA laddering. M=marker; lanes 1=control siRNA; lanes 2=Bcl-2(a) siRNA; lanes 3=Bcl-2(b) si-RNA. Cells were harvested for analysis 48 hr post-tranfection. d, Annexin V-positive apoptotic cells detected by FACS analysis (Methods). Cells were harvested at 24 and 48 hr post-tranfection as indicated. □=control siRNA; [symbol] =Bcl-2(a) siRNA; ▪=Bcl-2(b) siRNA; [symbol] =Bcl-2(b) anti-sense RNA. In all subsequent experiments Bcl-2(b) siRNA was employed to silence Bcl-2 expression, and apoptosis was confirmed by the independent techniques of DNA laddering and annexin V labelling with FACS analysis. e, Cytochrome c distribution in cells at the time of transfection (0 hr) and release into the cytosol of non-adherent cells collected 48 hr following transfection with Bcl-2 siRNA in HCT116 p53+/+ cells. P=pellet fraction; C=cytosolic fraction.

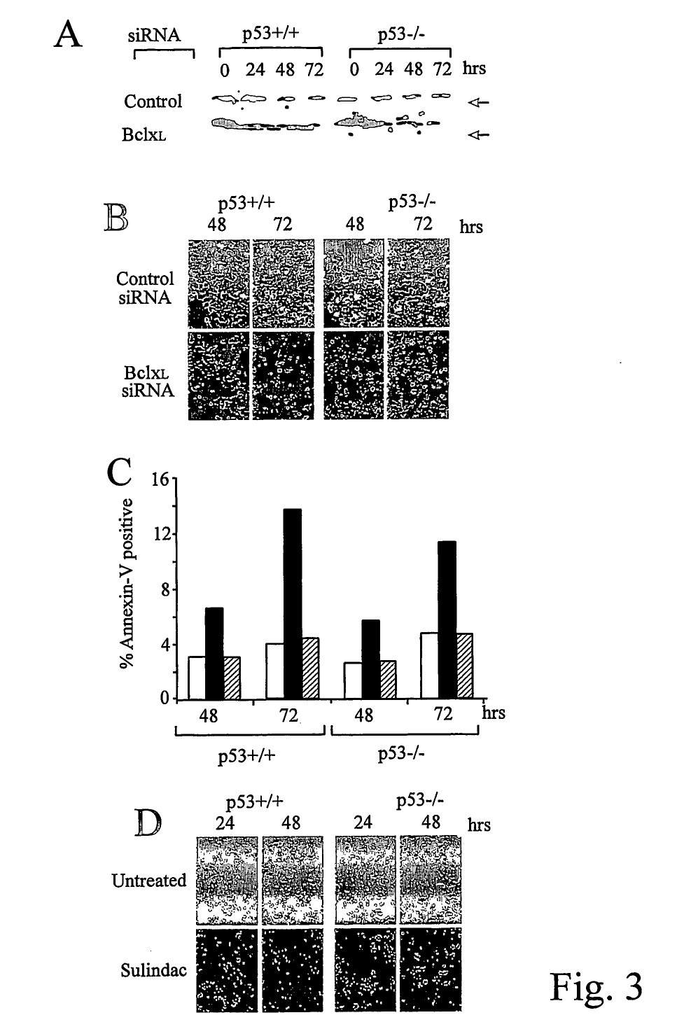

FIG. 3.

p53-independent apoptotic pathways in isogenic clones of HCT116 cells. a-c, siRNA silencing of the anti-apoptotic gene Bcl-x_{L} using the siRNA sequence shown in FIG. 1c. a, Immunoblot of BCl-x_{L} protein at different times after transfection with control siRNA or BCl-x_{L} siRNA. b, Phase contrast images of cells at 48 and 72 hr post-transfection with control siRNA or BCl-x_{L} siRNA. c, Early apoptotic cells detected by annexin V labelling and FACS analysis. Cells were harvested for analysis at 48 and 72 hr as indicated. □=control siRNA; ▪=Bcl-x_{L} siRNA; [symbol] =BC1-x_{L} anti-sense RNA. d, Apoptosis induced by treatment with sulindac Methods). Phase contrast images of cells at 24 and 48 hr post-treatment with sulindac, which activates Bax-dependent, p53-independent apoptosis (Zhang et al., 2000). Apoptosis was confirmed by DNA laddering and by FACS analysis of cells labelled with annexin V (not shown).

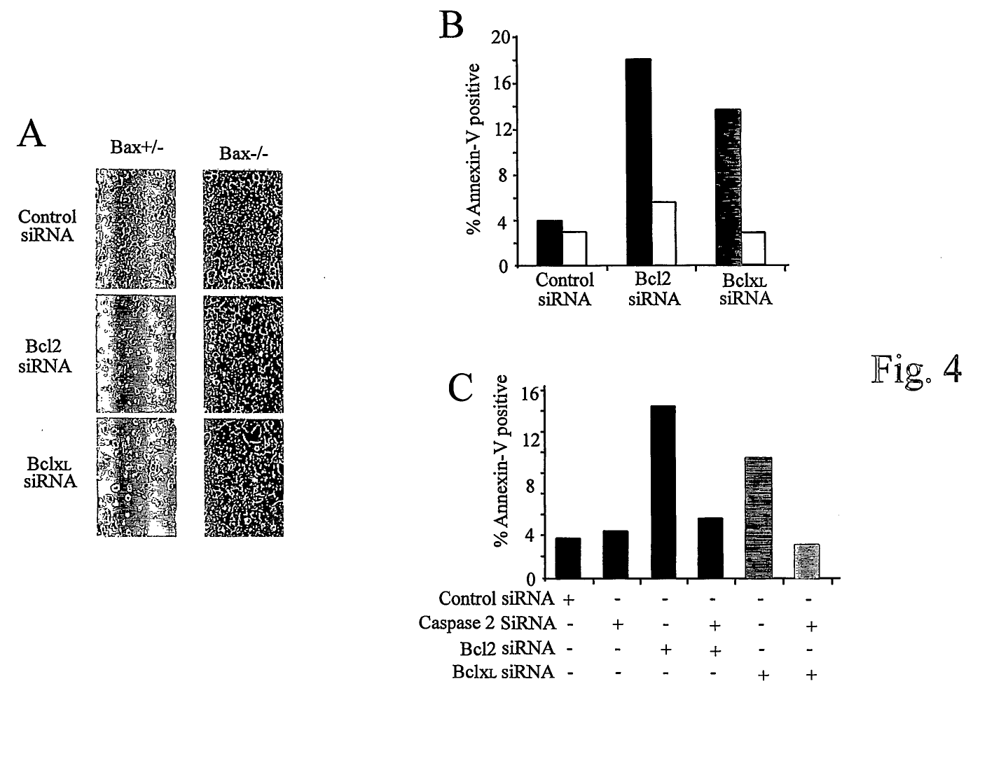

FIG. 4.

Apoptosis following silencing of Bcl-2 or BCl-x_{L} depends upon Bax and caspase 2. a, Phase contrast images of isogenic clones of Bax± and Bax−/− HCT116 cells at 72 hr post-transfection with control siRNA, with Bcl-2 siRNA or with BCl-x_{L} siRNA, as indicated. b, Apoptotic cells in isogenic clones of HCT116 cells detected by labelling with annexin V and FACS analysis. The cells were harvested at 72 hr post-transfection with control siRNA, Bcl-2 siRNA or BCl-x_{L} siRNA as indicated; =Bax ± cells and =Bax−/− cells. c, Apoptotic cells in HCT116 p53+/+ cells (the same clone as used in FIGS. 2 and 3) 72 hr following transfection with caspase 2 siRNA in combination with either Bcl-2 siRNA or Bcl-x_{L} siRNA as indicated.

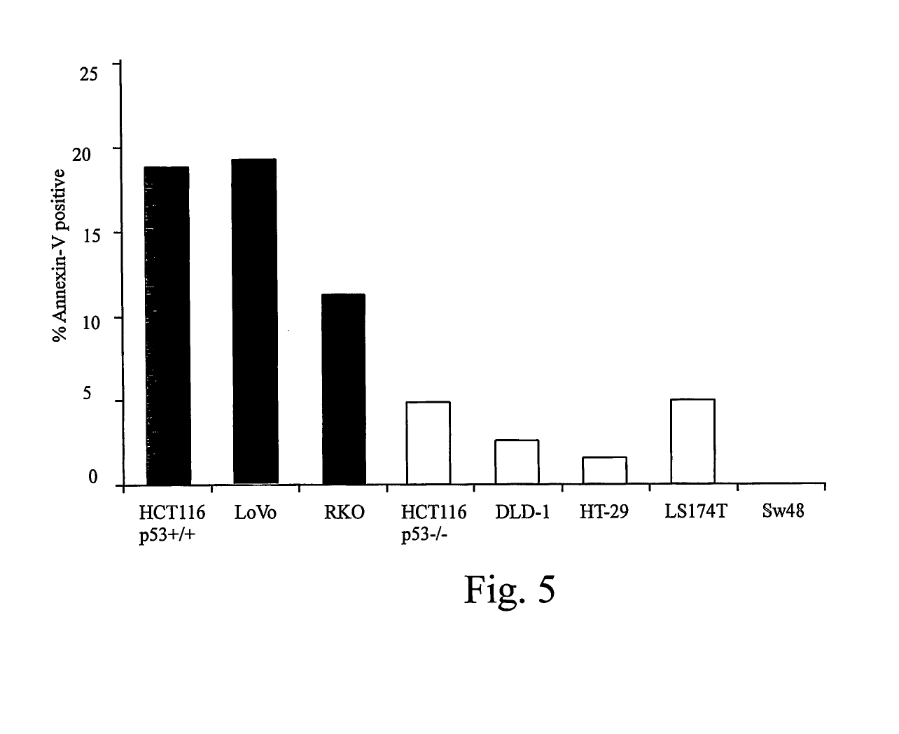

FIG. 5.

Apoptosis correlates with p53 status in individual human colorectal carcinoma cell lines following silencing of Bcl-2 expression.

Cells were transfected with Bcl-2 siRNA and apoptotic cells were determined after 48 hr (as described in FIG. 2 legend and methods). [symbol] =cell lines expressing endogenous wild -type p53; □=p53-deficient cell lines.

FIG. 6a and 6b illustrate the Bcl-2 alpha and Bcl-2 beta mRNA and protein sequence.

FIG. 7 illustrates the Bcl-x_{L} mRNA sequence

Selective silencing of Bcl-2 expression.

Paired isogenic clones of HCT116 p53+/+ and p53−/− cells (Bunz et al., 1999; Zhang et al., 2000) were used. To silence Bcl-2 expression we selected two Bcl-2 mRNA target sequences (FIG. 1a & b). Both are 100% conserved between human and murine Bcl-2. Silencing of Bcl-2 expression was monitored by immunoblotting the Bcl-2 protein. It should be noted that a previous, well controlled study failed to clearly detect Bcl-2 in immunoblots of HCT116 cell lysates (Zhang et al., 2000). This observation was confirmed when using the same antibody (N19, Santa Cruz; FIG. 1d). However, other antibodies clearly detect Bcl-2 in the HCT116 cells and, importantly, show that Bcl-2 protein levels are equivalent in the p53+/+ and p53−/− cells (FIG. 1e-h). The inevitable stress associated with the transfection process was not sufficient to activate p53 as a transcription factor in p53+/+ cells as evident from the absence of up-regulation of p21, a p53 target protein (Fig. li). The Bcl-2 protein fell to barely detectable levels within 24 h transfection with Bcl-2 siRNA (FIG. 2a). Interestingly, only one of the two Bcl-2 siRNAs silenced Bcl-2 expression (Bcl-2(b); FIG. 2) indicating that the mRNA sequence homologous to the non-effective siRNA (nucleotides 77 - 95; FIG. 1a) must somehow be protected from recognition and/or degradation by RNA interference. Such protection may arise due to localised mRNA secondary structure or protein-mRNA interactions (see Jiang and Milner, 2002). Control transfections included a random siRNA sequence (control siRNA, Jiang and Milner, 2002) and lamin A/C siRNA previously shown to suppress lamin A/C protein expression without inducing apoptosis (Elbashir et al., 2001).

Bcl-2 silencing induces p53-dependent apoptosis

By 48 h massive apoptosis was observed in the p53+/+ cells transfected with Bcl-2 siRNA (Bcl-2(b); FIG. 2b-d). Apoptosis in cells transfected with Bcl-2 anti-sense RNA (anti-sense sequence 354-372) was negligible (FIG. 2d) and equivalent to that observed for control siRNA. This confirms that apoptosis induced by Bcl-2 siRNA in p53+/+ cells is due to RNA interference. siRNA silencing of lamin A/C failed to induce apoptosis in either the p53+/+ or p53−/− cells (FIG. 2b). This demonstrates that the process of RNA interference per se is not sufficient to activate apoptosis in HCT116 p53+/+ cells. Unexpectedly the p53−/− cells failed to undergo apoptosis following silencing of Bcl-2 expression (FIG. 2b-d). Thus we conclude that selective silencing of Bcl-2 expression induces massive apoptosis of HCT116 colorectal cancer cells and that this effect is dependent upon p53.

It has recently been demonstrated that Bcl-2 can regulate an apoptotic pathway that is activated independently of mitochondrial cytochrome c release and Apaf-1/caspase 9 activation (Marsden et al., 2002). This raises the possibility that p53 may enable this cytochrome c-independent pathway, thus accounting for the observed differences in apoptosis between p53+/+ and p53−/− cells following silencing of Bcl-2 (FIG. 2). However, analysis of cytochrome c distribution clearly demonstrates release of cytochrome c into the cytosol in p53+/+ cells undergoing apoptosis following treatment with Bcl-2 siRNA (FIG. 2e). In long exposures of the immunoblots mitochondrial cytochrome c release was also evident in adherent p53+/+ cells treated with Bcl-2 siRNA whereas no cytosolic cytochrome c was detectable in parallel cultures of p53−/− cells treated with Bcl-2 siRNA (not shown). These results indicate that Bcl-2 silencing induces p53-dependent apoptosis via pathway(s) that involve the release of cytochrome c from the mitochondria.

BC1-x_{L} silencing induces p53-independent apoptosis.

The integrity of p53-independent apoptotic pathways was next confirmed by silencing the BCl-x_{L} gene, again using RNA interference. Bcl-x_{L} is an anti-apoptotic gene (Boise et al., 1993) and in colorectal epithelial cells a decrease in the ratio of Bcl-x_{L}:Bax is sufficient to induce apoptosis (Zhang et al., 2000). Therefore we predicted that selective silencing of BCl-x_{L} should induce apoptotic cell death in both p53+/+ and p53−/− cells. Indeed this proved to be the case. First we ascertained that the selected Bcl-x_{L} siRNA sequence (see FIG. 1c) effectively reduces Bcl-x_{L} protein expression (FIG. 3a), and then demonstrated its capacity to induce apoptosis (FIG. 3b-c). BCl-x_{L} protein levels declined between 24 and 48 hr post-transfection with Bcl-x_{L} siRNA and subsequently apoptosis was observed in both the p53+/+ and p53−/− cells. This demonstrates that p53 is not required for Bcl-x_{L}-regulated apoptotic pathway(s) in colorectal epithelial cells. Further verification of p53-independent apoptotic pathways was obtained by treating the cells with sulindac which is known to activate Bax-dependent apoptosis (Zhang et al., 2001). Sulindac induced apoptosis in both p53+/+ and p53−/− cells (FIG. 3d; see also Zhang et al., 2000). On the basis of these overall results we conclude that the observed lack of apoptosis in p53−/− cells treated with Bcl-2 siRNA (FIG. 2) cannot be attributed to either loss of Bax or other apoptotic pathway suppressed by BCl-x_{L}. This is consistent with the isogenic nature of the two cell clones and indicates that p53 is a selective requirement for apoptosis induced by Bcl-2 silencing.

Bax and caspase 2 are required for apoptosis following silencing of Bcl-2 or Bcl-x_{L}.

Thus far our results indicate that Bcl-2 constitutively suppresses apoptosis in colorectal cancer cells grown in culture and that, following silencing of Bcl-2 expression, the process of apoptosis requires p53. This is novel and places a pro-apoptotic function of p53 under Bcl-2 regulation. Moreover, this pro-apoptotic function of p53 does not require treatment of cells with cytotoxic agents such as 5-FU. (Note that the process of RNA interference by itself is not sufficient to activate p53-induced apoptosis, as demonstrated by lack of apoptosis in p53+/+ cells treated with lamin A/C siRNA, see above). In addition, we show that silencing of Bcl-x_{L} induces apoptosis in a p53-independent manner (FIG. 3). This is consistent with previous work identifying Bax as a major player in the apoptotic response of colorectal cancer cells, (Ionov et al., 2000; Zhang et al., 2001; LeBlanc et al., 2002) and BCl-x_{L} as its anti-apoptotic counterpart (when expressed exogenously, Zhang et al., 2001). These combined observations led us to reason that Bcl-2/p53 and Bcl-x_{L}/Bax might represent functional partners governing apoptosis in human colorectal epithelial cells. Within this scenario at least two putative apoptotic pathways might be envisaged: (i) Bcl-2/p53 may define an apoptotic pathway that is essentially independent of Bcl-x_{L}/Bax; or (ii) Bcl-2/p53 and Bcl-x_{L}/Bax may govern inter-related transitions in the apoptotic process. To discriminate between these two alternatives we silenced, individually, Bcl-2 and Bcl-x_{L} expression in isogenic clones of Bax± and Bax−/− HCT116 cells (note that the apoptotic response of Bax ± cells is equivalent to Bax+/+ cells; Zhang et al., 2000). siRNA silencing of Bcl-2 and of Bcl-x_{L} induced massive apoptosis in Bax ± cells but failed to induce significant apoptosis in Bax−/− cells (FIG. 4c and d). This clearly demonstrates that Bax is required for apoptosis in both Bcl-2-regulated and Bcl-x_{L}-regulated pathways.

The above results demonstrate that in colorectal carcinoma cells the Bcl-2 and BCl-x_{L} cell death pathways share commonalities in their requirement for Bax, but differ in their requirements for p53. It is possible that p53 is required to prine a pro-apoptotic pathway which is selectively suppressed by Bcl-2, thus lowering the apoptotic threshold consequent to Bcl-2 silencing. To further dissect the functional links between Bcl-2, Bcl-x_{L}, p53 and Bax we next asked if caspase 2 is also involved. Apoptosis induced by Bcl-2 or by Bcl-x_{L} silencing was blocked when caspase 2 siRNA was co-transfected with either Bcl -2 siRNA or BCl-x_{L} siRNA respectively (FIG. 4c; see Lassus et al, 2002 for caspase 2 siRNA sequence). siRNA silencing of caspase 2 alone (FIG. 4c), or transfection with anti-sense caspase 2 RNA (not shown), had no apparent effect on cell viability. Overall these results demonstrate that both Bax and caspase 2 are required for apoptosis following silencing of either Bcl-2 or Bcl-x_{L} in p53+/+ colorectal cancer cells. This is consistent with recent evidence that caspase 2 enables translocation of Bax into the mitochondria and subsequent mitochondrial membrane permeabilisation marked by release of cytochrome c (Marsden et al., 2002).

Effects of Bcl-2 siRNA on individual colorectal carcinoma cell lines of varying p53 status.

The above experiments involve isogenic clones of HCT116 cells and are thus tightly controlled for genetic variation. To establish the generality of our observations we silenced Bcl-2 in other human colorectal carcinoma cell lines, also defective for DNA mismatch repair and with defined p53 status (see Methods). In each case the presence of wild type p53 correlated with induction of apoptosis detectable 48 hours post-transfection with Bcl-2 siRNA, whereas p53-deficiency correlated with background levels of apoptosis (FIG. 5). These results confirm our observations with isogenic clones of p53+/+ and p53−/− HCT116 cells and are consistent with concept that Bcl-2 constitutively suppresses p53-dependent apoptosis in colorectal cancer cells.

Materials and Methods

Cell Lines and Transfections

HCT116 clones were cultured in DMEM with 10% FCS. All the cell clones were cultured with penicillin 100 units ml⁻¹ and streptomycin 100 μg ml⁻¹ at 37° C. in 5% CO₂ in air. Other human colorectal cancer cells lines, also defective for DNA mismatch repair (see Branch et al., 1995), were:- LoVo and RKO (p53 wild type); DLD1, LS174T, SW48 and HT29 (all p53 mutant). For transfection the cells were trypsinised and sub-cultured into 6 well plates (10 cm²) without antibiotics, 1.5×10⁵ cells per well. Selected 21-nucleotide RNAs were synthesised and BPLC purified (MWG; Germany) and annealed into siRNA duplexes according to the instructions supplied. 24 h after sub-culture the cells were transfected with siRNA formulated into liposomes (Oligofectamine, Life Technologies) according to the manufacturer's instructions. The protocol includes a short incubation in serum-free medium but controls demonstrated that this was not sufficient to activate a p53 response (see Results section). siRNA concentration was 0.58 μg per 1.5×10⁵ cells per well. The final volume of culture medium was 1.5 ml per well. Cells were harvested for analysis at various times thereafter as indicated in the results. Each experiment with HCT116 cells was carried out four or more times. Transfection efficiencies were established by transfecting with liposomes containing FITC-dextran (Jiang and Milner, 2002). Anti-sense RNA controls were included in each experiment using the respective anti-sense sequences for Bcl-2(b), Bcl-x_{L} and caspase 2 (see FIG. 1a. and text).

Immunoblotting and Mitochondrial Cytochrome c Release

For immunoblotting the transfected cells were trypsinised, washed in PBS and an aliquot removed for cell counting. The remaining cells were lysed in 50 μl lysis buffer (150 mM NaCl; 0.5% NP40; 50 mM Tris pH 8.0) on ice for 30 min. Samples were diluted 1:1 in 4×strength Laemlli's buffer. Proteins were resolved by 15% SDS-PAGE and electroblotted onto nitrocellulose membrane for antibody detection. Molecular weight markers and purified recombinant human p53 were included as markers (not shown). The following antibodies were employed: for Bcl-2=N19 and C-2 (Sant Cruz); Ab-1 and Ab-2 (Oncogene; note that Ab-1 gave non-specific background with multiple cellular proteins, not shown); and BD (Pharmingen) (FIG. 1b). The C-2 antibody gave the cleanest results and was subsequently used throughout this work. Lamin A/C=antibody 636; Santa Cruz); Bcl-x_{L}=Bcl-X antibody (Pharmingen; this antibody gave a relatively high non-specific background). P53=DO-1 antibody (Oncogene) and caspase 2=caspase-2L antibody (F-7; Santa Cruz). Visualisation of bound antibodies was by enhanced chemiluminescence (Roche). Cell fractionations and cytochrome c determinations were carried out as described in Marsden et al (2002, supplementary information).

Cell Growth, Cell Cycle Analysis and Apoptosis

Cell growth curves were determined by cell counting. Induction of apoptosis byi sulindac (see FIG. 3) employed sulindac sulphide 120 μM (Calbiochem). For cell cycle analysis the cells were harvested, washed with PBS and fixed in 90% ethanol overnight at -20° C. The fixed cells were pelleted, washed in PBS and resuspended in PBS containing 0.1 μg/ml propidium iodide with 200 U/ml RNase A and analysed by FACS. Apoptotic cells were identified using annexin-V-Fluos (Boehringer) following the manufacturer's protocol. Apoptosis was also verified by DNA laddering using the Suicide-track DNA ladder isolation kit (Oncogene) according to the manfacturer's instructions.

Discussion

In the present study we have used isogenic cell clones and siRNA to obtain defined combinations of pro- and anti-apoptotic gene expression in cells that are otherwise genetically equivalent. Our observations indicate a new cell death pathway in which Bcl-2 constitutively suppresses p53-dependent apoptosis. Apoptosis can also be induced by treating the cells with agents such as 5′ FU to activate p53 (Zhang et al., 2000 and results not shown). This is consistent with established evidence that activated p53 functions up-stream of Bcl-2 in response to genotoxic stress (see Strasser et al., 1994; and reviews by Johnstone et al, 2002; Cory and Adams 2002). To accommodate our present observations within the context of previous studies we suggest that Bcl-2 constitutively suppresses a novel pro-apoptotic function of p53 and that exposure to genotoxic stress over-rides Bcl-2 suppression by inducing the transactivation potential of p53. Once activated as a transcription factor p53 has the capacity to alter the expression ratios of Bcl-2 and BC1-x_{L} (down-regulated) and Bax (up-regulated) in favour of apoptosis (see Johnstone et al., 2002). From a clinical point of view this has proved very useful for anti-cancer therapy but carries the inherent risk of non-specific cytotoxicity and genotoxicity caused by p53-activating agents.

The present invention shows that p53 possesses pro-apoptotic properties that appear to be constitutively active, albeit suppressed by Bcl-2. Bcl-2 is identified as a potential and promising target for anti-cancer therapy for colorectal cancer (see also Reed et al., 2002) and the accessibility of Bcl-2 for siRNA silencing is demonstrated. The survival of other epithelial tumours may be similarly susceptible to Bcl-2 silencing. With the development of RNA interference the selective silencing of specific genes is now a realistic possibility, and the continual inventive evolution of targeted delivery systems (see, for example, Hood et al., 2002) should enable application of RNA interference to prevent and to treat cancer.

The present invention also carries important implications for patients with inherited DNA mismatch repair deficiencies and associated pre-disposition to colorectal cancer. In particular it argues against the use of sulindac as chemo-preventative in such patients since it is well established that defective mismatch repair renders the Bax gene susceptible to mutation and favours clonal expansion of Bax-deficient cells. In the present study we demonstrate that Bax is an essential mediator of apoptosis regulated by the newly discovered Bcl-2/p53 pathway (see above). It follows that, in patients with mismatch repair defects, any selective pressure for Bax-deficient cells may exacerbate tumorogenesis and should be avoided. The constitutive pro-apoptotic function of p53 may be linked with apical apoptosis in the normal colorectal epithelium. If so, failure of apoptosis in colorectal epithelial tumours might reflect inappropriate suppression of intrinsic p53-induced apoptosis.

A strong candidate in this regard is Bcl-2 which constitutively blocks p53-induced apoptosis and enables the survival of colorectal cancer cells. Such a model is consistent with the late onset of p53 mutation in the malignant progression of colorectal cancer. It also re-enforces Bcl-2 as a prime target for development of novel anti-cancer agents.

Viral homologues of cellular anti-apoptotic genes such as Bcl-2 represent promising targets for the treatment of viral-induced cancers. The silencing of human Bcl-2 expression identifies a Bcl-2 mRNA sequence that is accessible for the RNAi machinery. Only one of two different anti-Bcl-2 siRNAs has been found to be effective, underscoring the importance of target sequence selection and verification during the development of any anti-viral therapy based upon RNA interference. Viral homologues of Bcl-2 (v-Bcl-2) containing viral-specific nucleotide sequences accessible for RNAi will represent promising targets for RNAi-based anti-viral/anti-cancer therapies.

### REFERENCES

Boise, L. H. et al. 1993. Bcl-x, a bcl-2-related gene that functions as a dominant regulator of apoptotic cell death. Cell 74, 597-608.
Branch, P., Hampson, R. and Karran, P. 1995. DNA mismatch binding defects, DNA damage tolerance, and mutator phenotype in human colorectal,carcinoma cell lines. Cancer Research 55, 2304-2309.
Bunz, F. et al. 1999. Disruption of p53 in human cancer cells alters the responses to chemotherapeutic agents. J. Clin. Invest 104, 263-269.
Cory, S. and Adams, J. M. 2002. The Bcl2 family: regulators of the cell life-or-death switch. Nature Reviews Cancer 2, 647-656.
Elbashir, S. M. et al. 2001. Duplexes of 21-nucleotide RNAs mediate RNA interference in cultured mammalian cells. Nature 441, 494-498.
He, T. C., Chan, T. A., Vogelstein, B. & Kinzler, K. W. 1999. PPARδ is an APC-regulated target of nonsteroidal anti-inflammatory drugs. Cell 99, 335-345.
Hood, J. D. et al. 2000. Tumor regression by targeted gene delivery to the neovasculature. Science 296, 2404-2407.
Ionov, Y., Peinado, M. A., Malkhosyan, S., Shibita, D. & Perucho, M. 1993. Ubiquitous somatic mutations in simple repeated sequences reveal a new mechanism for colon carcinogenesis. Nature 363, 558- 561.
Ionov, Y., Yamamoto, H., Krajewsli, S., Reed, J. C. & Perucho, M. 2000. Mutational inactivation of the pro-apoptotic gene Bax confers selective advantage during tumour clonal evolution. Proc. Natl. Acad. Sci. USA 97, 10872-10877.
Jiang, M. & Milner, J. 2002. Selective silencing of viral gene expression in HPV-positive human cervical carcinoma cells treated with siRNA, a primer of RNA interference. Oncogene 21: 6041-6048
Lassus, P., Optiz-Araya, X. & Lazebnik, Y. 2002. Requirement for caspase 2 in stress-induced apoptosis before mitochondrial permeabilisation. Science 297: 1352-1354.
Johnstone, R. W., Ruefli, A. A. & Lowe, S. W. 2002. Apoptosis: a link between cancer genetics and chemotherapy. Cell 108: 153-164.
LeBlanc, H. et al. 2002. Tumor-cell resistance to death receptor-induced apoptosis through mutational inactivation of the proapoptotic Bcl-2 homologue Bax. Nature Medicine 8, 274-281.
Lynch, H. T. 1999. Hereditary nonpolyposis colorectal cancer (HNPCRC). Cytogenet Cell Genet. 86, 130-135.
Marsden, V. S. et al. 2002. Apoptosis initiated by Bcl-2-regulated caspase activation independently of the cytochrome c/Apaf-1/caspase 9 apoptosome. Nature 419: 634-637.
Rampino, N. et al. 1997. Somatic frameshift mutations in the BAX gene in colon cancers of the microsatellite mutator phenotype. Science 275, 967-969.
Reed, J. C. 2002. Apoptosis-based therapies. Nat. Rev. Drug Discov. 1: 111-121. Strasser, A., Harris, A. W., Jacks, T. & Cory, S. 1994. DNA damage can induce apoptosis in proliferating lymphoid cellsvia p53-independent mechanisms inhibitable by BCL-2. Cell 79: 329-339.
Yamamoto, Y., Yin, M. J., Lin, K. M. & Gaynor, R. B. 1999. Sulindac inhibits activation of the NF-kappaB pathway. J. Biol. Chein. 274, 27307-27314.
Zhang, L., Yu, J., Park, B. H., Knizler, K. W. & Vogelstein, B. 2000. Role of BAX in the apoptotic response to anticancer agents. Science 290: 989-992.

1. A method of regulating apoptosis, said method comprising introducing into a cell an RNA construct comprising a nucleotide sequence which is homologous to mRNA within said cell, wherein said mRNA includes genetic information of a gene element involved in the regulation of apoptosis.

2. A method according to claim 1 wherein said gene element is involved in the suppression of apoptosis.

3. A method according to claim 1 wherein said gene element is Bcl-2.

4. A method according to claim 3 wherein the gene element comprises a nucleic acid molecule, or part thereof, selected from the group consisting of:
(i) a nucleic acid molecule as represented by FIGS. 6A (SEQ ID NO: 7) or 6B (SEQ ID NO:9) or a functional fragment thereof;
(ii) a nucleic acid molecule which hybridises to any of the nucleic acid sequences in (i) and which has siRNA activity;
(iii) a nucleic acid molecule which is degenerate as a result of the genetic code to the nucleic acid sequence of (i) and/or (ii) above.

5. A method according to claim 1 wherein said gene element is Bcl-x_{L}.

6. A method according to claim 5 wherein the gene element comprises a nucleic acid molecule, or part thereof, selected from the group consisting of:
(i) a nucleic acid molecule as represented by FIG. 7 (SEQ ID NO:11) or a functional fragment thereof;
(ii) a nucleic acid molecule which hybridises to any of the nucleic acid sequences in (i) and which has siRNA activity;
(iii) a nucleic acid molecule which is degenerate as a result of the genetic code to the nucleic acid sequence of (i) and/or (ii) above.

7. A method according to claim 1 wherein said gene element is a viral homologue of a gene involved in the regulation of apoptosis.

8. An siRNA construct having a nucleotide sequence which is homologous to mRNA transcribed from a gene element involved in the regulation of apoptosis.

9. An siRNA construct according to claim 8 wherein said construct is from 15 to 25 nucleotides in length.

10. An siRNA construct according to claim 9 wherein said construct is from 19 to 23 nucleotides in length.

11. An siRNA construct of claim 8 comprising;
(i) a nucleotide sequence that is homologous to a part or fragment of the nucleic acid sequences in FIG. 6A (SEQ ID NO:7) or FIG. 6B (SEQ ID NO:9);
(ii) a nucleotide sequence which is degenerate as a result of the genetic code to the nucleic acid sequence of (i) above.

12. An siRNA construct according to claim 8 comprising a nucleotide sequence that is homologous to Bcl-2 mRNA nucleotides 354-372 of FIG. 6a (SEQ ID NO:7).

13. An siRNA according to claim 8 comprising;
(i) a nucleotide sequence that is homologous to a part or fragment of the nucleic acid sequence in FIG. 7 (SEQ ID NO:11);
(ii) a nucleotide sequence which is degenerate as a result of the genetic code to the nucleic acid sequence of (i) above.

14. An siRNA construct according to claim 8 comprising a nucleotide sequence that is homologous to Bcl-xL nucleotides 347-366 of FIG. 7 (SEQ ID NO: 11).

15. A method of treating a disease or condition associated with inappropriate apoptosis comprising administering to a subject an RNA construct wherein said RNA construct has a nucleotide sequence which is homologous to mRNA present within a cell of said subject and wherein said mRNA includes genetic information of a gene element involved in the regulation of apoptosis.

16. The method of claim 15, wherein said construct comprises
(i) a nucleotide sequence that is homologous to a part or fragment of the nucleic acid sequences in FIG. 6A (SEQ ID NO:7) FIG. 6B (SEQ ID NO:9) or FIG. 7 (SEQ ID NO: 11):
(ii) a nucleotide sequence which is degenerate as a result of the genetic code to the nucleic acid sequence of (i) above.

17. A pharmaceutical composition comprising an RNA construct according to claim 8 and a pharmaceutically acceptable excipient.

18. The method of claim 15, wherein the RNA construct induces apoptosis.

19. The method of claim 15, wherein the disease is colorectal cancer.

20. The method of claim 15, wherein the disease is a viral induced cancer.


SEQUENCE LISTING (11 sequences)

SEQ ID NO: 1 - DNA, 21 bp, Artificial Sequence
  Bcl-2 small interfering RNA sequence (siRNA)
ggggcuacgagugggaugctt

SEQ ID NO: 2 - DNA, 21 bp, Artificial Sequence
  Bcl-2 small interfering RNA sequence (siRNA)
ttccccgaugcucacccuacg

SEQ ID NO: 3 - DNA, 21 bp, Artificial Sequence
  Bcl-2 small interfering RNA sequence (siRNA)
gcugcaccugacgcccuuctt

SEQ ID NO: 4 - DNA, 21 bp, Artificial Sequence
  Bcl-2 small interfering RNA sequence (siRNA)
ttcgacguggacugcgggaag

SEQ ID NO: 5 - DNA, 21 bp, Artificial Sequence
  Bcl-xL small interfering RNA sequence (siRNA)
cagggacagcauaucagagtt

SEQ ID NO: 6 - DNA, 21 bp, Artificial Sequence
  Bcl-xL small interfering RNA sequence (siRNA)
ttgucccugucguauagucuc

SEQ ID NO: 7 - DNA, 720 bp, Homo sapiens
atggcgcacgctgggagaacagggtacgataaccgggagatagtgatgaagtacatccattataagctgtcgcagaggggctacgagtgggatgcgggagatgtgggcgccgcgcccccgggggccgcccccgcgccgggcatcttctcctcgcagcccgggcacacgccccatacagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccaggcagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtga

SEQ ID NO: 8 - protein, 239 aa, Homo sapiens
MAHAGRTGYDNREIVMKYIHYKLSQRGYEWDAGDVGAAPPGAAPAPGIFSSQPGHTPHTAASRDPVARTSPLQTPAAPGAAAGPALSPVPPVVHLTLRQAGDDFSRRYRRDFAEMSRQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 9 - DNA, 618 bp, Homo sapiens
atggcgcacgctgggagaacggggtacgacaaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgcgacccggtcgccaggacctcgccgctgcagaccccggctgcccccggcgccgccgcggggcctgcgctcagcccggtgccacctgtggtccacctggccctccgccaagccggcgacgacttctcccgccgctaccgcggcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcatctggtgatgtgagtctgggctga

SEQ ID NO: 10 - protein, 205 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLALRQAGDDFSRRYRGDFAEMSSQLHLTPFTARGRFATVVEELFRDGVNWGRIVAFFEFGGVMCVESVNREMSPLVDNIALWMTEYLNRHLHTWIQDNGGWVGASGDVSLG

SEQ ID NO: 11 - DNA, 702 bp, Homo sapiens
atgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga